(19) United States
(12) Patent Application Publication
(10) Pub. No.: US 2020/0115765 A1
(43) Pub. Date: Apr. 16, 2020

(54) IN SITU DETECTION OF MULTIPLE PREDOMINANT SPECIES OF ASIAN CARP

(71) Applicant: The USA, as represented by the DOI, Washington, DC (US)

(72) Inventor: Christopher Merkes, La Crosse, WI (US)

(73) Assignee: The United States of America, as represented by the secretary of the Department of the Interior, Washington, DC (US)

(21) Appl. No.: 16/157,565

(22) Filed: Oct. 11, 2018

(51) Int. Cl. C12Q 1/6888

(52) U.S. Cl. CPC C12Q 1/6888; C12Q 2600/124

(57) ABSTRACT

The invention uses the loop-mediated isothermal amplification (LAMP) method of DNA amplification to detect the presence of Asian Carp, including the following species: Hypophthalmichthys nobilis, H. molitrix, Ctenopharyngodon idella, Mylopharyngodon piceus. The LAMP primers disclosed are designed to anneal to a mitochondrial DNA sequence only found in Asian Carp to initiate replication of that segment of DNA.

This invention allows minimally trained users to test water samples in under an hour rather than manually screening thousands of fish that could be difficult to identify visually.

### INCORPORATION OF SEQUENCE LISTING

Incorporated by reference in its entirety herein is a computer-readable nucleotide sequence listing submitted concurrently herewith and identified as follows: One 3,419 Byte ASCII (Text) file named “USGS_17-010_SEQ.txt,” created on Oct. 3, 2018.

### FIELD OF INVENTION

This invention relates to the field of nucleic acid products used in the analysis of nucleic acids, such as primers or probes for detection or identification of organisms and more specifically to an assay for testing for Asian Carp.

### BACKGROUND OF THE INVENTION

The U.S. Geological Survey (USGS) is a bureau of the Department of the Interior with a mission to provide time-sensitive scientific data about the nation's geological resources as crises are occurring. The role of the USGS is critical to support federal agencies in protecting lakes, rivers and tributaries from the effects of Asian Carp. Asian Carp are prohibited invasive species which cause irreversible ecosystem damage and potentially render the environment inhospitable to the $7 billion per year fishing industry. Introduction of Asian Carp into an ecosystem can eradicate native fish populations in a matter of years.

In 2017, the USGS began working with U.S. Fish and Wildlife biologists and law enforcement agencies to develop rapid in situ test kits to detect the presence of prohibited species such as Asian Carp.

Several states have passed regulations to control human commercial activity that can introduce Asian carp into previously unaffected areas, such as transporting bait. Despite these measures, Asian Carp are expected to spread to 31 states by the end of 2019. Multiple agencies have collaborated to control the spread of Asian Carp.

There are currently ten species of Asian Carp on the government's prohibited species list. On this list, the most prevalent species is the grass carp; silver carp ranks fourth, bighead carp ranks sixth, and black carp ranks seventh.

In 2015, a published study concluded that 1 out of 550 Midwestern bait shops tested positive for genetic material from Asian Carp.

PCR tests are known in the art, which can test for Asian Carp DNA.

It is a problem known in the art that PCR testing is expensive. These tests must be conducted in a professional laboratory and require a series of alternating temperature steps or cycles. The equipment required to conduct the PCR testing is costly, and testing cannot be conducted on site.

Recently developed loop-mediated isothermal amplification (LAMP) tests can be run on-site at one constant temperature without the need for expensive equipment.

There is an unmet need for rapid, on-site testing methods that can be used to detect multiple species of Asian Carp.

### SUMMARY OF THE INVENTION

The invention uses the loop-mediated isothermal amplification (LAMP) method of DNA amplification to detect the presence of Asian Carp, including the following species: Hypophthalmichthys nobilis, H. molitrix, Ctenopharyngodon idella, Mylopharyngodon piceus.

The LAMP primers disclosed are designed to anneal to mitochondrial DNA sequences only found in Asian Carp to initiate replication of that segment of DNA. As the replication continues, a fluorescent dye intercalates into the double-stranded DNA products and fluoresces at a known wavelength. This fluorescent light is detected by the any instrument used to analyze the samples. An increase in fluorescent signal intensity over time indicates the presence of the target DNA sequence.

### BRIEF DESCRIPTION OF THE DRAWINGS

FIG. 1 illustrates loop-mediated isothermal amplification (LAMP) assay apparatus 100 comprised of novel primers and reagents to detect the presence of Asian Carp.

FIG. 2 illustrates exemplary primer concentrations and effective ranges for each LAMP primer.

FIG. 3 is a table of the common DNA sequence carried by Asian Carp and detected by a LAMP test apparatus and corresponding method.

FIG. 4 illustrates an exemplary method for using LAMP testing apparatus 100 to detect the presence of Asian Carp DNA.

### TERMS OF ART

As used herein, the term “LAMP apparatus” means any test which detects DNA and which does not require multiple cycles of temperature changes for amplification.

As used herein, the term “potential degenerate base sequence” means a sequence position that is either specifically defined as one nucleotide base or which can be one of two bases. If a primer sequence includes a degenerate base, it is shown as R for purine bases or Y for pyrimidine bases. Purine bases include guanine or adenine, meaning that an R in the primer sequence represents either a G or an A. Pyrimidine bases include cytosine or thymine, meaning that a Y in the primer sequence represents either a C or a T.

### DETAILED DESCRIPTION OF THE INVENTION

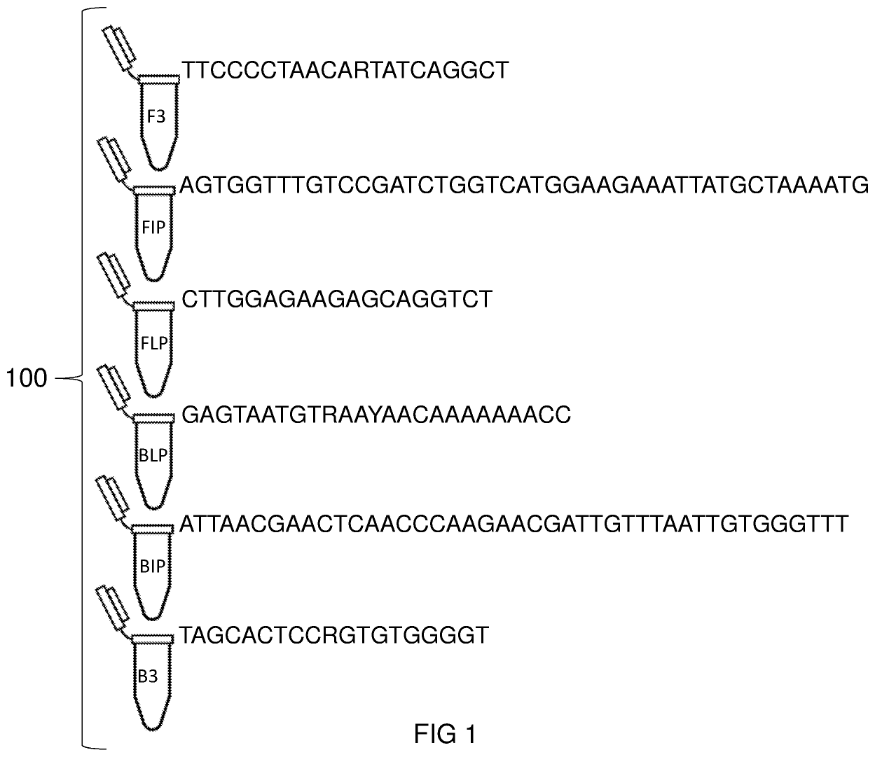

FIG. 1 illustrates loop-mediated isothermal amplification (LAMP) assay apparatus 100 comprised of novel primers and reagents to detect the presence of Asian Carp. As illustrated, LAMP Assay apparatus includes the forward inner primer (FIP), the forward loop primer (FLP), the backward loop primer (BLP), the backward inner primer (BIP), and the backward primer (B3) having novel primer sequences designed to bind specifically to four target Asian Carp species.

LAMP apparatus 100 further includes reagents, which include but are not limited to enzymes, pH buffers, triphosphate dNTPs, magnesium salt, and dyes known in the art to replicate the target DNA sequence and dye the replicated DNA to make the copies visible to a machine. LAMP assay apparatus 100 can utilize any LAMP assay result detection method known in the art. In various embodiments, LAMP assay apparatus 100 can dye replicated DNA copies to make them visible to the human eye.

In the embodiment shown, primers, enzymes, and dyes are combined with a sample in a reaction tube and incubated at a constant temperature for a user-determined period of time. The sample may either include an unknown number of copies of the target DNA sequence, or a known number of copies of the target DNA sequence.

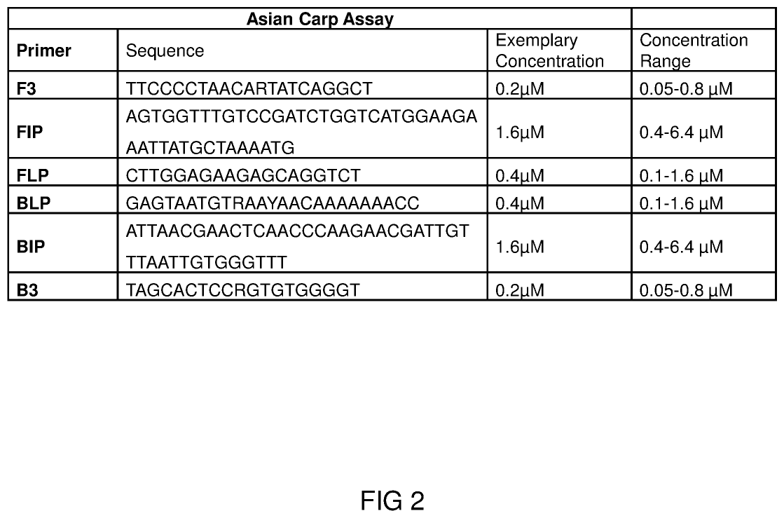

FIG. 2 illustrates exemplary primer concentrations and effective ranges for each LAMP primer. Concentrations of each LAMP reaction component (including primers, enzyme, pH buffers, triphosphate dNTPs, magnesium salt, dye, and sample) in the reaction tube, may vary based on test conditions and equipment. However, alternate concentration ranges may be used. In the exemplary embodiment shown, the reaction concentration of F3 is between approximately 0.05 and 0.8 μM. The reaction concentration of FIP is between approximately 0.4 and 6.4 μM. The reaction concentration of FLP is between approximately 0.1 and 1.6 μM. The reaction concentration of BLP is between approximately 0.1 and 1.6 μM. The reaction concentration of BIP is between approximately 0.4 and 6.4 μM. The reaction concentration of B3 is between approximately 0.05 and 0.8 μM.

In the exemplary embodiment shown, some of the primer sequence positions can have one of two bases, known as degenerate bases. If a primer sequence includes a degenerate base, it is shown as R for purine bases or Y for pyrimidine bases. Purine bases include guanine or adenine, meaning that an R in the primer sequence represents either a G or an A. Pyrimidine bases include cytosine or thymine, meaning that a Y in the primer sequence represents either a C or a T.

In the exemplary embodiment shown, the primers listed in FIG. 1 bind to Asian Carp DNA to cause replication at a constant temperature of approximately 55 to 70 degrees Celsius, with an optimal temperature of 62.9 degrees Celsius.

In the exemplary embodiment shown, the oligonucleotide primers detect Asian Carp species, including but not limited to the following species Hypophthalmichthys nobilis, H. molitrix, Ctenopharyngodon idella, and Mylopharyngodon piceus. These species are commonly known as Bighead Carp, Silver Carp, Grass Carp, and Black Carp, respectively.

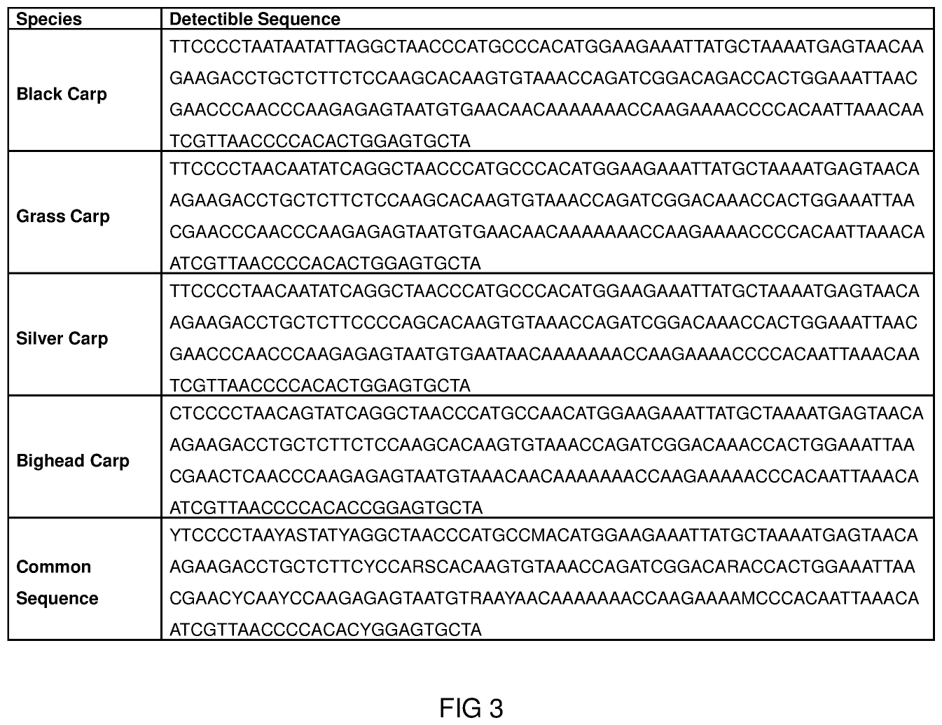

FIG. 3 is a table of the common DNA sequence carried by Asian Carp and detected by (LAMP) assay apparatus 100 and corresponding method.

The table in FIG. 2 illustrates the detected DNA sequence carried by the Black Carp (Mylopharyngodon piceus), Grass Carp (Ctenopharyngodon Idella), Silver Carp (Hypophthalmichthys molitrix), and Bighead Carp (H. nobilis), which are detected by LAMP assay apparatus 100. These sequences are listed in the text file as <SEQ ID NO. 1>, <SEQ ID NO. 2>, <SEQ ID NO. 3>, and <SEQ ID NO. 4>, respectively. The common sequence illustrates the similarities between the four sequences. Positions with variations are marked with R, Y, S, and M. R represents both purine bases and includes adenine (A) and guanine (G). Y represents both pyrimidine bases and includes cytosine (C) and Thymine (T). S represents the strong bases cytosine (C) and guanine (G). M represents the amino bases adenine (A) and cytosine (C).

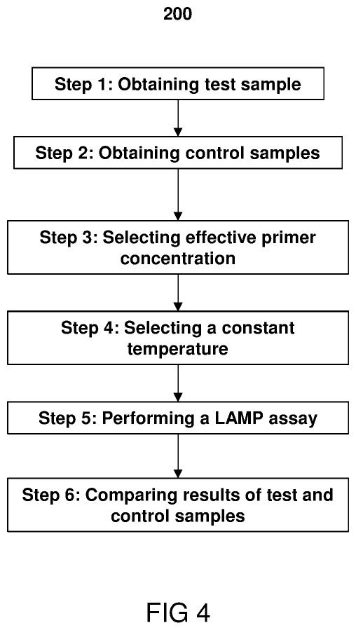

FIG. 4 illustrates exemplary method 200 for using LAMP testing apparatus 100 to detect the presence of Asian Carp DNA.

Step 1 is the step of obtaining a test sample to be tested for the presence of Asian Carp genetic material.

In the exemplary embodiment, a test sample is a quantity of DNA extracted from sampled water that may have contained Asian Carp. This step may include any method known in the art for extracting DNA from a water sample. Assay apparatus 100 detects Asian Carp genetic material in the test sample, whether or not the fish is still present in the sampled container at the time of obtaining the sampled water. In various embodiments, a single fish sheds enough DNA to be detected by the test.

In various embodiments, LAMP assay apparatus 100 can detect a single Silver Carp in a pool of 33,000 flathead minnows.

In still other embodiments, LAMP assay apparatus 100 may detect as few as 10 DNA copies per 40 μL LAMP reaction.

Step 2 is the step of obtaining a positive control sample and negative control sample for comparison. In various embodiments, a positive control sample may be included in LAMP assay apparatus 100.

In all embodiments, a positive control sample has at least a minimum number of Asian Carp DNA copies. In various embodiments, the minimum number of DNA copies may be 10 DNA copies per 40 μL LAMP reaction.

Using LAMP assay apparatus 100 on this positive control sample will result in a fluorescent signal. The negative control sample has no Asian Carp DNA copies and using LAMP assay apparatus 100 on this negative control sample will not result in a fluorescent signal. After using LAMP assay apparatus 100 on the test samples and control samples, the results will be compared to determine which test samples resulted in a fluorescent signal, indicating that the test samples contain Asian Carp DNA.

In various embodiments, the positive control sample will indicate that the LAMP reagents (reaction components) are working properly. If the reaction with the positive control sample does not show a fluorescent signal, then the LAMP reagents may need to be replaced.

In various embodiments, a fluorescent signal in the negative control reaction will indicate that the assay has resulted in false positives and needs to be repeated with replaced reagents.

Step 3 is the step of selecting an effective concentration of each of the following primers.

In one exemplary embodiment, the LAMP primer set binds to Asian Carp DNA and includes the following sequences (as illustrated in FIGS. 1 and 2):

a forward primer having the following sequence: 5′-TTCCCCTAACARTATCAGGCT-3′ <SEQ ID NO. 5> or <SEQ ID NO. 6>;

a forward inner primer having the following sequence: 5′-AGTGGTTTGTCCGATCTGGTCATGGAAGAAATTATGCTAAAATG-3′ <SEQ ID NO. 7>;

a backward primer having the following sequence 5′-TAGCACTCCRGTGTGGGGT-3′ <SEQ ID NO. 8> or <SEQ ID NO. 9>;

a backward inner primer having the following sequence 5′-ATTAACGAACTCAACCCAAGAACGATTGTTTAATTGTGGGTTT-3′ <SEQ ID NO. 10>;

a forward loop primer having the following sequence 5′-CTTGGAGAAGAGCAGGTCT-3′ <SEQ ID NO. 11>; and

a backward loop primer having the following sequence 5′-GAGTAATGTRAAYAACAAAAAAACC-3′ <SEQ ID NO. 12>, <SEQ ID NO. 13>, <SEQ ID NO. 14>, or <SEQ ID NO. 15>.

In the exemplary embodiment shown in FIG. 1, the effective reaction concentration for each primer is approximately 0.2 to 1.6 μM. In various embodiments, the reaction concentration for each primer may be approximately 0.05 to 6.4 μM.

Step 4 is the step of selecting a constant temperature.

In the exemplary embodiment, the constant LAMP reaction temperature is approximately 55 to 70° C. In various embodiments, the optimal constant LAMP reaction temperature is 62.9° C.

Step 5 is the step of performing a LAMP assay at a constant LAMP reaction temperature of approximately 55 to 70° C. In various embodiments, the optimal constant LAMP reaction temperature is 62.9° C.

To perform the LAMP assay, each test sample, positive control sample, and negative control sample is placed in a separate reaction tube. Each reaction tube also receives enzymes and dyes known in the art and the novel primer set. The reaction tubes are incubated at the constant temperature chosen in Step 5. The user determines the time period for incubation based on manufacturer instruction. The concentration of each sample and each commercially available component in the reaction tube is determined by the user, according to manufacturer recommendations. The reaction concentration of each primer was determined by the user in Step 4.

The invention uses loop-mediated isothermal amplification of DNA. The LAMP primers anneal to a segment of Asian Carp mitochondrial DNA to initiate replication of the segment. As the replication continues, a fluorescent dye intercalates into the double-stranded DNA products and fluoresces at a known wavelength. This fluorescent light is detected by any instrument used to analyze the samples, and increasing fluorescent signal intensity over time indicates the presence of the target DNA.

In various embodiments, various commercially available enzymes, buffers, coenzymes, adjuvants, dyes, and deoxyribonucleic acids can be used to run assay apparatus 100. One exemplary embodiment may utilize the commercially available reagent Isothermal Master Mix (ISO-004) from Pro Lab Diagnostics.

Step 6 is the step of comparing the result of each test sample to the result of the positive and negative control samples to determine if a minimum threshold is exceeded. In various embodiments, using assay apparatus 100 on the positive control sample will result in a fluorescent signal. Using assay apparatus 100 on a test sample with a number of Asian Carp DNA copies that exceeds the minimum threshold will result in a fluorescent signal significantly more intense than the intensity of the background fluorescent signal, as determined by the user. In various embodiments, the minimum detectible threshold is 10 copies per 40 μL LAMP reaction. In various embodiments, the fluorescent signal is detectable by a machine.

What is claimed is:

1. An assay testing apparatus, comprised of:
a set of primers comprised of
a forward primer;
a forward inner primer;
a forward loop primer;
a backward primer;
a backward inner primer; and
a backward loop primer;
wherein each of said primers binds to a common DNA sequence
YTCCCCTAAYASTATYAGGCTAACCCATGCCMACATGGAAGAAATTATGC
TAAAATGAGTAACAAGAAGACCTGCTCTTCYCCARSCACAAGTGTAAACC
AGATCGGACARACCACTGGAAATTAACGAACYCAAYCCAAGAGAGTAATG
TRAAYAACAAAAAAACCAAGAAAAMCCCACAATTAAACAATCGTTAACCC
CACACYGGAGTGCTA
to indicate the presence of one or more species of Asian Carp.

2. The apparatus of claim 1, wherein two or more of said six primers are a potential degenerate base sequence.

3. The apparatus of claim 1, wherein said one or more species of Asian Carp is selected from a group consisting of Hypophthalmichthys nobilis, H. molitrix, Ctenopharyngodon Idella, and Mylopharyngodon piceus.

4. The apparatus of claim 1, wherein said common DNA sequence is selected from a group consisting of <SEQ ID NO. 1>, <SEQ ID NO. 2>, <SEQ ID NO. 3>, and <SEQ ID NO. 4>.

5. The apparatus of claim 1, which further includes a positive control having a known number of copies of said common DNA sequence.

6. The apparatus of claim 1, wherein said set of six primers is comprised of:
a forward primer having the sequence 5′-TTCCCCTAACARTATCAGGCT-3′ selected from a group consisting of <SEQ ID NO. 5> and <SEQ ID NO. 6>;
a forward inner primer having the sequence 5′-AGTGGTTTGTCCGATCTGGTCATGGAAGAAATTATGCTAAAATG-3′ <SEQ ID NO. 7>;
a backward primer having the sequence 5′-TAGCACTCCRGTGTGGGGT-3′ selected from a group consisting of <SEQ ID NO. 8> and <SEQ ID NO. 9>;
a backward inner primer having the sequence 5′-ATTAACGAACTCAACCCAAGAACGATTGTTTAATTGTGGGTTT-3′ <SEQ ID NO. 10>;
a forward loop primer having the sequence 5′-CTTGGAGAAGAGCAGGTCT-3′ <SEQ ID NO. 11>; and
a backward loop primer having the sequence 5′-GAGTAATGTRAAYAACAAAAAAACC-3′ selected from a group consisting of <SEQ ID NO. 12>, <SEQ ID NO. 13>, <SEQ ID NO. 14>, and <SEQ ID NO. 15>.

7. The apparatus of claim 6, wherein R is selected from a group consisting of A and G.

8. The apparatus of claim 6, wherein Y is selected from a group consisting of C and T.

9. The apparatus of claim 6, wherein said forward primer and said backward primer each have an effective concentration of approximately 0.2 μM.

10. The apparatus of claim 6, wherein said forward inner primer and said backward inner primer each have an effective concentration of approximately 1.6 μM.

11. The apparatus of claim 6, wherein said forward loop primer and said backward loop primer each have an effective concentration of approximately 0.4 μM.

12. The apparatus of claim 6, wherein said set of six primers binds to said common DNA sequence at a constant temperature of approximately 62.9 degrees Celsius.

13. The apparatus of claim 6, wherein said set of six primers has a minimum detection limit of approximately 10 gene copies per 40 μL reaction.

14. A method for making a LAMP assay panel apparatus for detecting Asian Carp genetic material, comprised of the steps of:
selecting one or more target species from a group consisting of Hypophthalmichthys nobilis, H. molitrix, Ctenopharyngodon idella, and Mylopharyngodon piceus;
selecting a common DNA sequence carried by said one or more target species; and
selecting a forward primer sequence, a forward inner primer sequence, a forward loop sequence, a backward primer sequence, a backward inner primer sequence, and a backward loop sequence, wherein each of said primer sequences corresponds to said common DNA sequence.

15. The method of claim 14, which further includes the step of selecting said common DNA sequence from a group consisting of <SEQ ID NO. 1>, <SEQ ID NO. 2>, <SEQ ID NO. 3>, and <SEQ ID NO. 4>.

16. The method of claim 15, which further includes the step of selecting 5′-TTCCCCTAACARTATCAGGCT-3′ as said forward primer sequence, 5′-AGTGGTTTGTCCGATCTGGTCATGGAAGAAATTATGCTAAAAT G-3′ as said forward inner primer sequence, 5′-TAGCACTCCRGTGTGGGGT-3′ as said backward primer sequence 5′-ATTAACGAACTCAACCCAAGAACGATTGTTTA ATTGTGGGTTT-3′ as said backward inner primer sequence 5′-CTTGGAGAAGAGCAGGTCT-3′ as said forward loop primer sequence and 5′-GAGTAATGTRAAYAACAAAAAAACC-3′ as said backward loop primer sequence.

17. A method for detecting the genetic material of invasive aquatic species, comprised of the steps of:
obtaining DNA extracted from at least one sample of water which potentially contains more than 10 copies of a common DNA sequence carried by a species of Asian Carp;
preparing a LAMP primer set which binds to the genetic material of one or more Asian Carp species, wherein said LAMP primer set is comprised of a forward primer, a forward inner primer, a backward primer, a backward inner primer, a forward loop primer, and a backward loop primer;
selecting a constant temperature range of 62 to 63 degrees Celsius;
performing a LAMP assay for detecting an Asian Carp gene at said constant temperature range, by adding said LAMP primer set to said DNA extracted from at least one sample of water; and
detecting if said DNA extracted from at least one sample of water contains at least 10 copies of said common DNA sequence.

18. The method of claim 17, which further includes the steps of:
creating a positive control with at least 10 copies of said consensus DNA sequence; and
performing said LAMP assay by adding said LAMP primer set to said positive control.

19. The method of claim 17, which further includes the step of selecting 5′-TTCCCCTAACARTATCAGGCT-3′ as a forward primer sequence, 5′-AGTGGTTTGTCCGATCTGGTCATGGAAGAAATTATGCTAAAAT G-3′ as a forward inner primer sequence, 5′-TAGCACTCCRGTGTGGGGT-3′ as a backward primer sequence, 5′-ATTAACGAACTCAACCCAAGAACGATTGTTTA ATTGTGGGTTT-3′ as a backward inner primer sequence, 5′-CTTGGAGAAGAGCAGGTCT-3′ as a forward loop primer sequence and 5′-GAGTAATGTRAAYAACAAAAAAACC-3′ as a backward loop primer sequence.

20. The method of claim 19, which further includes the steps of:
adding said forward primer to said at least one sample to achieve an effective concentration of approximately 0.2 μM and said backward primer to said at least one sample to achieve an effective concentration of approximately 0.2 μM;
adding said forward inner primer to said at least one sample to achieve an effective concentration of approximately 1.6 μM and said backward inner primer to said at least one sample to achieve an effective concentration of approximately 1.6 μM; and
adding said forward loop primer to said at least one sample to achieve an effective concentration of approximately 0.4 μM and said backward loop primer to said at least one sample to achieve an effective concentration of approximately 0.4 μM.


SEQUENCE LISTING (15 sequences)

SEQ ID NO: 1 - DNA, 215 bp, Mylopharyngodon piceus
ttcccctaataatattaggctaacccatgcccacatggaagaaattatgctaaaatgagtaacaagaagacctgctcttctccaagcacaagtgtaaaccagatcggacagaccactggaaattaacgaacccaacccaagagagtaatgtgaacaacaaaaaaaccaagaaaaccccacaattaaacaatcgttaaccccacactggagtgcta

SEQ ID NO: 2 - DNA, 215 bp, Ctenopharyngodon idella
ttcccctaacaatatcaggctaacccatgcccacatggaagaaattatgctaaaatgagtaacaagaagacctgctcttctccaagcacaagtgtaaaccagatcggacaaaccactggaaattaacgaacccaacccaagagagtaatgtgaacaacaaaaaaaccaagaaaaccccacaattaaacaatcgttaaccccacactggagtgcta

SEQ ID NO: 3 - DNA, 214 bp, Hypophthalmichthys molitrix
ttcccctaacaatatcaggctaacccatgcccacatggaagaaattatgctaaaatgagtaacaagaagacctgctcttccccagcacaagtgtaaaccagatcggacaaaccactggaaattaacgaacccaacccaagagagtaatgtgaataacaaaaaaaccaagaaaaccccacaattaaacaatcgttaaccccacactggagtgcta

SEQ ID NO: 4 - DNA, 215 bp, Hypophthalmichthys nobilis
ctcccctaacagtatcaggctaacccatgccaacatggaagaaattatgctaaaatgagtaacaagaagacctgctcttctccaagcacaagtgtaaaccagatcggacaaaccactggaaattaacgaactcaacccaagagagtaatgtaaacaacaaaaaaaccaagaaaaacccacaattaaacaatcgttaaccccacaccggagtgcta

SEQ ID NO: 5 - DNA, 21 bp, Asian Carp
ttcccctaacagtatcaggct

SEQ ID NO: 6 - DNA, 21 bp, Asian Carp
ttcccctaacaatatcaggct

SEQ ID NO: 7 - DNA, 44 bp, Asian Carp
agtggtttgtccgatctggtcatggaagaaattatgctaaaatg

SEQ ID NO: 8 - DNA, 19 bp, Asian Carp
tagcactccggtgtggggt

SEQ ID NO: 9 - DNA, 19 bp, Asian Carp
tagcactccagtgtggggt

SEQ ID NO: 10 - DNA, 43 bp, Asian Carp
attaacgaactcaacccaagaacgattgtttaattgtgggttt

SEQ ID NO: 11 - DNA, 19 bp, Asian Carp
cttggagaagagcaggtct

SEQ ID NO: 12 - DNA, 25 bp, Asian Carp
gagtaatgtaaacaacaaaaaaacc

SEQ ID NO: 13 - DNA, 25 bp, Asian Carp
gagtaatgtgaacaacaaaaaaacc

SEQ ID NO: 14 - DNA, 25 bp, Asian Carp
gagtaatgtgaacaacaaaaaaacc

SEQ ID NO: 15 - DNA, 25 bp, Asian Carp
gagtaatgtgaataacaaaaaaacc